(19) United States
(12) Patent Application Publication
(10) Pub. No.: US 2025/0332589 A1
(43) Pub. Date: Oct. 30, 2025

(54) MICROFLUIDIC DEVICE AND PRODUCTION METHOD THEREFOR

(71) Applicant: AGC Inc., Tokyo (JP)

(72) Inventor: Amandeep JINDAL, Tokyo (JP)

(73) Assignee: AGC Inc., Tokyo (JP)

(21) Appl. No.: 19/263,882

(22) Filed: Jul. 9, 2025

Related U.S. Application Data
(63) Continuation of application No. PCT/JP2024/002260, filed on Jan. 25, 2024.

(30) Foreign Application Priority Data
Feb. 24, 2023 (JP) 2023-026913

(51) Int. Cl. B01L 3/00

(52) U.S. Cl. CPC B01L 3/502707; B01L 2200/12; B01L 2300/0819; B01L 2300/0887; B01L 2300/12

(57) ABSTRACT

This microfluidic device includes a laminate having a porous layer laminated on a substrate. A production method for the microfluidic device includes spinning fibers having a core-shell structure using an electrospinning method and depositing the fibers on the substrate to form the porous layer.

### CROSS-REFERENCE TO RELATED APPLICATIONS

The present application is a continuation application of International application No. PCT/JP2024/002260, filed on Jan. 25, 2024, which claims the priority of on Japanese Patent Application No. 2023-026913, filed Feb. 24, 2023, the content of which is incorporated herein by reference.

### BACKGROUND OF THE INVENTION

### Field of Invention

The present invention relates to a microfluidic device and a production method therefor.

### Description of Related Art

Microfluidic devices which utilize technology in which electrowetting techniques or the like are used to move liquid droplets are already known. In Patent Document 1, in order to achieve liquid droplet movement, a dielectric parylene C layer is formed by a CVD method on the top of a plurality of electrodes so as to cover the electrodes, and a layer of Teflon (a registered trademark) is then formed on the parylene C layer by a spin-coating method.

### CITATION LIST

### Patent Document

Patent Document 1: Japanese Unexamined Patent Application, First Publication No. 2022-20665

### SUMMARY OF INVENTION

However, the production process for the microfluidic device disclosed in Patent Document 1 is complex.

Further, although the Teflon layer formed by spin-coating exhibits excellent hydrophobicity, the liquid repellency is not entirely satisfactory. In order to facilitate control of the movement of the liquid droplets, further improvements in the liquid repellency would be desirable.

The present invention provides a microfluidic device having excellent liquid repellency, and a production method that enables a microfluidic device having excellent liquid repellency to be produced using a simple process.

### Solution to Problem

The present invention provides a microfluidic device and a production method therefor that include the following aspects [1] to [20].

[1] A microfluidic device including a laminate having a porous layer laminated on a substrate.

[2] The microfluidic device according to [1], wherein the porous layer is a layer of a nonwoven fabric.

[3] The microfluidic device according to [2], wherein the nonwoven fabric is a nonwoven fabric formed using an electrospinning method.

[4] The microfluidic device according to [2] or [3], wherein a fiber constituting the nonwoven fabric contains a fluororesin.

[5] The microfluidic device according to [4], wherein the fiber that constitutes the nonwoven fabric further contains a non-fluororesin.

[6] The microfluidic device according to any one of [2] to [5], wherein the fiber that constitutes the nonwoven fabric has a core-shell structure.

[7] The microfluidic device according to [6], wherein one of core and shell of the core-shell structure contains a fluororesin, and the other contains a non-fluororesin.

[8] The microfluidic device according to [4], [5] or [7], wherein the fluororesin is an amorphous fluororesin.

[9] The microfluidic device according to [4], [5], [7] or [8], wherein the fluororesin contains a polymer having a unit having a fluorine-containing aliphatic cyclic structure that constitutes the main chain.

[10] The microfluidic device according to [9], wherein the unit having a fluorine-containing aliphatic cyclic structure that constitutes the main chain is at least one type of unit selected from the group consisting of units formed by cyclopolymerization of a diene-based fluorine-containing monomer, and units based on a cyclic fluorine-containing monomer.

[11] The microfluidic device according to [5] or [7], wherein the non-fluororesin is a crystalline non-fluororesin.

[12] The microfluidic device according to [5] or [7], wherein the non-fluororesin is at least one type of resin selected from the group consisting of polycaprolactone, polyethylene glycol, polyvinyl alcohol, polyacrylonitrile, polyaniline, acrylic resins, polyurethane, polystyrene, polycarbonate, polyvinylpyrrolidone, chitosan, gelatin and cellulose acetate.

[13] The microfluidic device according to any one of [1] to [12], wherein the device is a biochip.

[14] A production method for a microfluidic device, the method including spinning fibers having a core-shell structure using an electrospinning method and depositing the fibers on a substrate to form a porous layer.

[15] The production method according to [14], wherein one of the core and the shell of the core-shell structure contains a fluororesin, and the other contains a non-fluororesin.

[16] The production method according to [15], wherein the fluororesin is an amorphous fluororesin.

[17] The production method according to [15] or [16], wherein the fluororesin contains a polymer having a unit having a fluorine-containing aliphatic cyclic structure that constitutes the main chain.

[18] The production method according to [17], wherein the unit having a fluorine-containing aliphatic cyclic structure that constitutes the main chain is at least one type of unit selected from the group consisting of units formed by cyclopolymerization of a diene-based fluorine-containing monomer, and units based on a cyclic fluorine-containing monomer.

[19] The production method according to any one [15] of to [18], wherein the non-fluororesin is a crystalline non-fluororesin.

[20] The production method according to any one of [15] to [18], wherein the non-fluororesin is at least one type of resin selected from the group consisting of polycaprolactone, polyethylene glycol, polyvinyl alcohol, polyacrylonitrile, polyaniline, acrylic resins, polyurethane, polystyrene, polycarbonate, polyvinylpyrrolidone, chitosan, gelatin and cellulose acetate.

### Advantageous Effects of Invention

The microfluidic device of the present invention exhibits excellent liquid repellency.

The production method for a microfluidic device according to the present invention enables the production of a microfluidic device with excellent liquid repellency using a simple process.

### BRIEF DESCRIPTION OF DRAWINGS

FIG. 1 is a schematic cross-sectional view illustrating one example of a microfluidic device.

FIG. 2 is a schematic structural diagram illustrating one example of an electrospinning device used in the production of a nonwoven fabric composed of fibers having a core-shell structure.

### DETAILED DESCRIPTION OF THE INVENTION

Meanings and definitions of the terminology used in the present invention are as follows.

An “aliphatic cyclic structure” means a saturated or unsaturated cyclic structure that has no aromaticity.

A “fluorine-containing aliphatic cyclic structure” means an aliphatic cyclic structure in which a fluorine atom or a fluorine-containing group is bonded to each of at least a portion of the carbon atoms that constitute the main skeleton of the ring. Examples of fluorine-containing groups include perfluoroalkyl groups, perfluoroalkoxy groups, and ═CF₂. Other substituents besides a fluorine atom or a fluorine-containing group may also be bonded to a portion of the carbon atoms that constitute the main skeleton of the ring.

An “etheric oxygen atom” is a single oxygen atom that exists between two carbon atoms (—C—O—C—).

The “weight average molecular weight” describes a polymethyl methacrylate (hereinafter sometimes abbreviated as PMMA) equivalent value measured by gel permeation chromatography (hereinafter also abbreviated as GPC).

In this description, a group represented by formula 2 is also referred to as a “group 2”, and a compound represented by formula ma1 is also referred to as a “compound ma1”. This naming convention also applies to groups and compounds and the like represented by other formulas.

The expression “a to b” used to indicate a numerical range means a range that includes the numerical values before and after the “to” as the lower limit and upper limit respectively.

In order to facilitate description, the dimensions of the various sections illustrated in the drawings may sometimes differ from the actual values.

### [Microfluidic Device]

The microfluidic device according to one embodiment of the present invention includes a laminate having a porous layer laminated on a substrate.

The laminate may have the porous layer laminated to one surface of the substrate, or may have porous layers laminated to both surfaces of the substrate. Further, the substrate and the porous layer may be in direct contact, or another layer may be interposed between the substrate and the porous layer.

With the exception that the above laminate constitutes at least a portion of the structural members of the microfluidic device, the microfluidic device according to this embodiment of the present invention may be similar to conventional microfluidic devices.

In the microfluidic device according to this embodiment, typically, at least a portion of the surface of the porous layer side of the laminate forms microchannels along which a liquid can flow, with the liquid flowing along these microchannels making contact with the porous layer. The liquid generally includes water.

The microfluidic device may be of a type in which liquid droplets flow along the microchannels, or of a type in which the liquid flows continuously along the microchannels. In terms of the fact that superior liquid repellency offers great usability, the microfluidic device is preferably of the type in which liquid droplets flow along the microchannels.

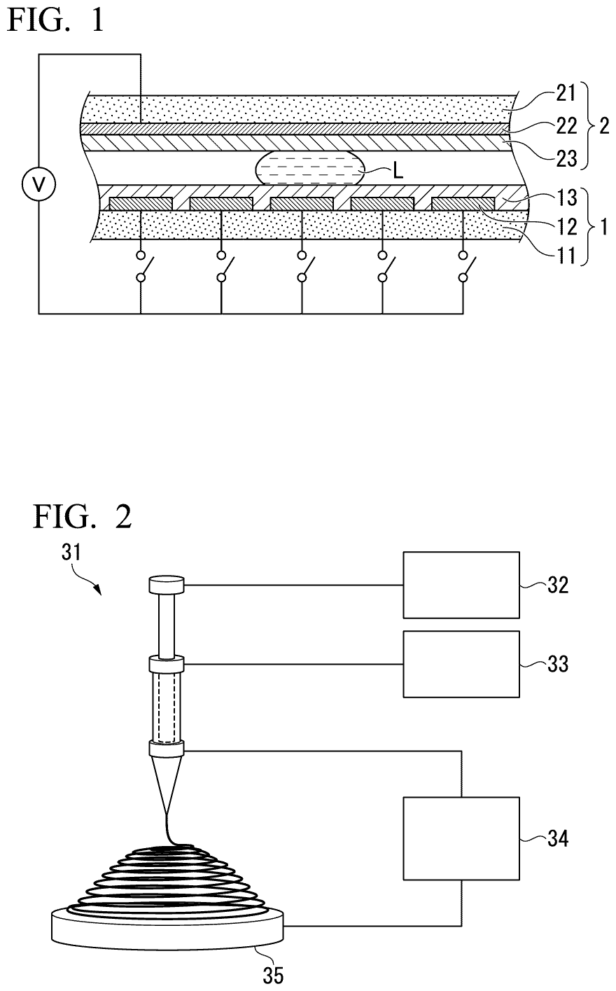

One example of a microfluidic device of the type in which liquid droplets flow along the microchannels is a digital microfluidic device in which driving of the liquid droplets is conducted by electrowetting on dielectric (EWOD), such as the device illustrated in FIG. 1. However, the microfluidic device of the present invention is not limited to this particular example.

The microfluidic device illustrated in FIG. 1 includes a first laminate 1 and a second laminate 2.

The first laminate 1 includes a first substrate 11 having a plurality of electrodes 12, and a first dielectric layer 13 provided on top of the first substrate 11 so as to cover the plurality of electrodes 12. The first dielectric layer 13 is a porous layer.

The second laminate 2 comprises a second substrate 21 having a common electrode 22, and a second dielectric layer 23 provided on top of the first substrate 11 so as to cover the common electrode 22. The second dielectric layer 23 may or may not be a porous layer.

The first laminate 1 and the second laminate 2 are arranged with the first dielectric layer 13 and the second dielectric layer 23 facing each other with a gap therebetween. The gap between the first laminate 1 and the second laminate 2 forms a microchannel through which a liquid droplet L can flow. The width of the gap, namely the distance between the first laminate 1 and the second laminate 2, is typically within a range from 100 to 300 μm.

The microfluidic device of FIG. 1 is typically used with the first laminate 1 positioned beneath the second laminate 2.

A dielectric layer that is not porous may be provided between the plurality of electrodes 12 and the first dielectric layer 13.

### (Porous Layer)

Laminating the porous layer on the substrate enables excellent liquid repellency to be achieved. The porous layer has a plurality of pores that form fine unevenness on the layer surface. It is thought that the lotus effect generated by this fine unevenness yields the superior liquid repellency. Further, because excellent liquid repellency can be imparted by simply laminating the porous layer to the substrate, the production process is simple.

In those cases where the microfluidic device is a digital microfluidic device such as that illustrated in FIG. 1, the porous layer is preferably a dielectric layer. A dielectric means a substance in which the development of a charge at the two ends of the substance (dielectricity) dominates over conductivity.

Examples of the porous layer include a fibrous layer or a layer of a porous material.

A fibrous layer is a layer composed of fibers, and examples include layers composed of compacted fibers such as nanofibers (for example, nonwoven fabrics), woven fabrics, and knitted materials. The fibers themselves may or may not be porous.

A porous material layer is a layer composed of a porous material. The porous material itself is porous, and examples include activated carbon, diatomaceous earth, and silica gels.

Among these possibilities, in terms of achieving superior flatness for the formed layer, the porous layer is preferably a layer of a nonwoven fabric.

Among nonwoven fabrics, in terms of providing particularly superior liquid repellency, a nonwoven fabric formed by the electrospinning method (also referred to as electrostatic spinning) is preferred. In the electrospinning method, a nonwoven fabric composed of fibers having a nano-order diameter can be produced using a simple operation. Further, a nonwoven fabric formed by the electrospinning method tends to exhibit a smaller pore size and more uniform pore size distribution when compared with nonwoven fabrics formed by other methods.

In terms of yielding superior liquid repellency, microbial adhesion prevention, and dielectric properties, the fibers used for forming the nonwoven fabric are preferably fibers that contain a resin. Examples of the resin include fluororesins and non-fluororesins. In terms of exhibiting excellent liquid repellency and microbial adhesion prevention, a fluororesin is preferred. A combination of a fluororesin and a non-fluororesin may also be used. These fluororesins and non-fluororesins are described below in further detail.

The fibers containing a resin may also contain additives such as antimicrobial agents, antistatic agents, flame retardants, and anti-aging agents.

In the fibers containing a resin, the amount of the resin, relative to the total mass of the resin-containing fibers, is preferably at least 20% by mass, more preferably at least 40% by mass, and may be 100% by mass.

The fibers that constitute the nonwoven fabric may have a uniform structure or a non-uniform structure through the radial direction of the fiber.

Examples of fibers having a uniform structure include fibers composed of a single type of resin, and fibers composed of a blended resin containing two or more types of resin. Examples of blended resins include blended resins of two or more types of fluororesin, blended resins of one or more types of fluororesin and one or more types of non-fluororesin, and blended resins of two or more types of non-fluororesin.

Examples of fibers having a non-uniform structure include fibers having a core-shell structure (hereinafter also referred to as “core-shell fibers”). Examples of these core-shell fibers include fibers in which one of the core or the shell contains a fluororesin, and the other contains a non-fluororesin, fibers in which the core and the shell contain different fluororesins, and fibers in which the core and the shell contain different non-fluororesins.

In terms of exhibiting more superior liquid repellency, the fibers that constitute the nonwoven fabric are preferably core-shell fibers, and fibers in which at least one of the core and the shell contains a fluororesin are more preferred. In terms of exhibiting superior moldability and production costs, fibers in which one of the core and the shell contains a fluororesin and the other contains a non-fluororesin are particularly preferred.

In those cases where the one of the core and the shell contains a fluororesin and the other contains a non-fluororesin, the proportion of the fluororesin relative to the total mass of the core-shell fibers is preferably within a range from 5 to 95% by mass, and more preferably from 10 to 90% by mass. Further, the proportion of the non-fluororesin relative to the total mass of the core-shell fibers is preferably within a range from 5 to 95% by mass, and more preferably from 10 to 90% by mass.

In those cases where the core contains a fluororesin and the shell contain a non-fluororesin, the proportion of the fluororesin relative to the mass of the core is preferably at least 20% by mass, more preferably at least 40% by mass, and may be 100% by mass. Further, the proportion of the non-fluororesin relative to the mass of the shell is preferably at least 20% by mass, more preferably at least 40% by mass, and may be 100% by mass.

In those cases where the shell contains a fluororesin and the core contain a non-fluororesin, the proportion of the fluororesin relative to the mass of the shell is preferably at least 20% by mass, more preferably at least 40% by mass, and may be 100% by mass. Further, the proportion of the non-fluororesin relative to the mass of the core is preferably at least 20% by mass, more preferably at least 40% by mass, and may be 100% by mass.

Although described below in further detail, the fluororesin is preferably an amorphous fluororesin. On the other hand, when the nonwoven fabric is formed using the electrospinning method, at least one of the core or the shell preferably contains a crystalline non-fluororesin. Accordingly, in those cases where the nonwoven fabric is composed of core-shell fibers formed by electrospinning, it is preferable that one of the core and the shell contains an amorphous fluororesin, and the other contains a crystalline non-fluororesin.

The diameter of the fibers that constitute the nonwoven fabric is preferably within a range from 20 to 800 nm, more preferably from 100 to 700 nm, and particularly preferably from 300 to 600 nm. Provided the diameter of the fibers that constitute the nonwoven fabric is not more than the above upper limit, the liquid repellency is more superior, whereas provided the diameter is at least as large as the above lower limit, the mechanical strength is superior.

The diameter of the fibers can be determined using the method described below in the examples.

In those cases where the fibers that constitute the nonwoven fabric are core-shell fibers, the diameter of the core is preferably within a range from 2 to 760 nm, more preferably from 5 to 670 nm, and particularly preferably from 15 to 570 nm.

The thickness of the shell is preferably within a range from 14 to 399 nm, more preferably from 48 to 348 nm, and particularly preferably from 143 to 293 nm.

The diameter of the core can be determined using energy-dispersive X-ray spectroscopy.

The thickness of the shell can be determined from the formula: (fiber diameter-core diameter)/2.

In those cases where the fibers that constitute the nonwoven fabric are core-shell fibers, the ratio represented by shell mass/core mass (hereinafter also referred to as the “shell/core ratio”) is preferably within a range from 1/1 to 19/1, and more preferably from 2/1 to 9/1. Provided the shell/core ratio is at least as high as the above lower limit, the liquid repellency is more superior, whereas provided the shell/core ratio is not more than the above upper limit, the moldability is superior.

The thickness of the porous layer is preferably within a range from 5 to 900 μm, more preferably from 20 to 300 μm, and particularly preferably from 50 to 200 μm. Provided the thickness of the porous layer is at least as large as the above lower limit, the liquid repellency and the mechanical strength are more superior, whereas provided the thickness is not more than the above upper limit, the moldability and productivity are superior.

The thickness of the porous layer is measured using the method described below in the examples.

The water contact angle of the surface of the porous layer is preferably at least 100°, more preferably at least 115°, and particularly preferably 130° or higher. The water contact angle of the surface of the porous layer is preferably as high as possible, and although there is no particular limit on the upper limit, the water contact angle is typically not more than 160°

The water contact angle is an indicator of the water repellency, with a higher water contact angle indicating superior water repellency.

The water contact angle is measured using the method described below in the examples.

The surface roughness of the porous layer, expressed as the value of Sa (arithmetic mean height) measured using laser microscopy in accordance with the evaluation method prescribed in ISO 25178, is preferably within a range from 5 to 5,000 nm, more preferably from 20 to 1,000 nm, and particularly preferably from 50 to 500 nm. Provided the value of Sa (arithmetic mean height) falls within the above range, the liquid repellency is more superior.

The density of the porous layer is preferably within a range from 2 to 150 g/m³, more preferably from 5 to 100 g/m³, and particularly preferably from 7 to 80 g/m³. Provided the density falls within this range, the liquid repellency is more superior.

### <Fluororesin>

The fluororesin may be any fluororesin capable of forming a porous layer, and conventional fluororesins may be used.

Examples of preferred fluororesins include amorphous fluororesins. Amorphous fluororesins exhibit superior solvent solubility, meaning a solution can be used as a raw material, and therefore the porous layer can be formed more easily.

Examples of the amorphous fluororesins include polymers having a fluorine-containing aliphatic cyclic structure within the main chain (hereinafter also referred as “polymer A”). Because the polymer A has a fluorine-containing aliphatic cyclic structure within the main chain, absorption of ultraviolet radiation through to near infrared radiation is minimal and the transparency is excellent. Polymers having a fluorine-containing aliphatic cyclic structure within the main chain can also be dissolved in solvents. Further, the main chain is resistant to degradation, providing excellent weather resistance.

In the fluorine-containing aliphatic cyclic structure, the ring skeleton may be a carbon ring structure formed solely of carbon atoms, or may be a hetero ring structure containing one or more atoms other than carbon atoms in the ring structure (hereinafter, an atom other than a carbon atom is sometimes referred to as a “hetero atom”). Examples of the hetero atom include an oxygen atom or a nitrogen atom. The number of atoms constituting the ring skeleton of the fluorine-containing aliphatic cyclic structure is preferably within a range from 4 to 7 atoms, and particularly preferably from 5 to 6 atoms. In other words, the aliphatic cyclic structure is preferably a 4- to 7-membered ring, and more preferably a 5- or 6-membered ring.

In terms of yielding superior transparency and solvent solubility, the fluorine-containing aliphatic cyclic structure is preferably a fluorine-containing aliphatic cyclic structure with a hetero ring structure having an etheric oxygen atom within the ring skeleton, and a fluorine-containing aliphatic cyclic structure with a hetero ring structure having one or two etheric oxygen atoms within the ring skeleton is particularly desirable.

Examples of the fluorine-containing aliphatic cyclic structure include cyclic structures in which some or all of the hydrogen atoms in a hydrocarbon ring structure or hetero ring structure have each been substituted with a fluorine atom.

Among the various possibilities, fluorine-containing aliphatic cyclic structures in which some or all of the hydrogen atoms of a hetero ring structure having an etheric oxygen atom within the ring skeleton have each been substituted with a fluorine atom are preferred, and a fluorine-containing aliphatic cyclic structure in which some or all of the hydrogen atoms of a hetero ring structure having one or two etheric oxygen atoms within the ring skeleton have each been substituted with a fluorine atom is particularly desirable.

The fluorine-containing aliphatic cyclic structure is preferably a perfluoro aliphatic cyclic structure in which all of the hydrogen atoms in the hydrocarbon ring structure or hetero ring structure have been substituted with fluorine atoms.

The polymer A is preferably a polymer having a unit having a fluorine-containing aliphatic cyclic structure that constitutes the main chain (hereinafter also referred to as the “unit a1”). The unit a1 is preferably a perfluoro unit.

The expression that the fluorine-containing aliphatic cyclic structure “constitutes the main chain” means that at least one of the carbon atoms constituting the ring skeleton of the fluorine-containing aliphatic cyclic structure is a carbon atom that constitutes part of the main chain of the polymer. Because two carbon atoms derived from a polymerizable double bond constitute the main chain of the polymer, the above expression means that either one or two adjacent carbon atoms constituting the ring of the fluorine-containing aliphatic cyclic structure are carbon atoms derived from a single polymerizable double bond.

For example, in those cases where the unit a1 is formed by an addition polymerization of a monoene-based monomer, two carbon atoms derived from the polymerizable double bond constitute part of the main chain, and either those two carbon atoms are two adjacent carbon atoms within the ring skeleton, or one of the two carbon atoms is a carbon atom within the ring skeleton. Further, in those cases where the unit a1 is formed by a cyclopolymerization of a diene-based monomer, a total of four carbon atoms derived from the two polymerizable double bonds constitute part of the main chain, and two to four of those four carbon atoms are carbon atoms constituting the ring skeleton.

Examples of the unit a1 include units formed by cyclopolymerization of a diene-based fluorine-containing monomer, and units based on a cyclic fluorine-containing monomer. In either case, one of these units may be used alone, or a combination of two or more units may be used.

A diene-based fluorine-containing monomer is a monomer having two polymerizable double bonds and a fluorine atom. In the case of a diene-based fluorine-containing monomer, the unit a1 is formed by a cyclopolymerization. There are no particular limitations on the polymerizable double bond, but vinyl groups, allyl groups, acryloyl groups or methacryloyl groups are preferred. In these polymerizable double bonds, some or all of the hydrogen atoms bonded to carbon atoms may each be substituted with a fluorine atom.

A compound ma1 is preferred as the diene-based fluorine-containing monomer.

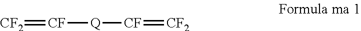

In the formula, Q represents a perfluoroalkylene group of 1 to 6 carbon atoms which may have an etheric oxygen atom and in which a portion of the fluorine atoms may each be substituted with a halogen atom other than a fluorine atom.

In the formula ma1, the number of carbon atoms in the perfluoroalkylene group for Q is typically within a range from 1 to 6, preferably from 1 to 5, and particularly preferably from 1 to 3. The perfluoroalkylene group is preferably either linear or branched, and a linear group is particularly desirable.

In the perfluoroalkylene group, a portion of the fluorine atoms may each be substituted with a halogen atom other than a fluorine atom. Examples of the halogen atom other than a fluorine atom include a chlorine atom or a bromine atom.

The perfluoroalkylene group may have an etheric oxygen atom.

A perfluoroalkylene group having an etheric oxygen atom is preferred as Q. In such cases, the etheric oxygen atom in the perfluoroalkylene group may exist at one terminal of the perfluoroalkylene group, etheric oxygen atoms may exist at both terminals of the perfluoroalkylene group, or the etheric oxygen atom may exist between carbon atoms of the perfluoroalkylene group. In terms of the cyclopolymerizability, the etheric oxygen atom preferably exists at one terminal of the perfluoroalkylene group.

Q is preferably a group q1 or a group q2.

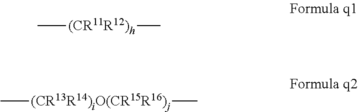

In these formulas, R¹¹, R¹², R¹³, R¹⁴, R¹⁵ and R¹⁶ each independently represent a fluorine atom, a chlorine atom, a trifluoromethyl group, or a trifluoromethoxy group. Further, h represents an integer of 2 to 4, and the plurality of R¹¹ and R¹² groups may be the same or different. Moreover, i and j each represent an integer of 0 to 3, provided that i+j is an integer of 1 to 3, and when i is 2 or 3, the plurality of R¹³ and R¹⁴ groups may be the same or different, and when j is 2 or 3, the plurality of R¹⁵ and R¹⁶ groups may be the same or different.

Furthermore, h is preferably 2 or 3, and it is preferable either that all of the R¹¹ and R¹² groups are fluorine atoms, or that all except one or two of the R¹¹ and R¹² groups are fluorine atoms. Moreover, it is preferable that i represents 0 and j represents either 1 or 2, and also preferable either that all of the R¹⁵ and R¹⁶ groups are fluorine atoms, or that all except one or two of the R¹⁵ and R¹⁶ groups are fluorine atoms.

Specific examples of the compound ma1 include the compounds shown below.

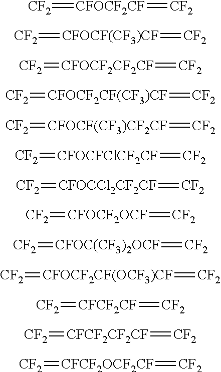

Examples of cyclic fluorine-containing monomers include monomers containing a fluorine-containing aliphatic ring, and having a polymerizable double bond between carbon atoms that constitute the fluorine-containing aliphatic ring, and monomers containing a fluorine-containing aliphatic ring, and having a polymerizable double bond between a carbon atom that constitutes part of the fluorine-containing aliphatic ring and a carbon atom outside of the fluorine-containing aliphatic ring.

A compound ma2 or a compound ma3 is preferred as the cyclic fluorine-containing monomer.

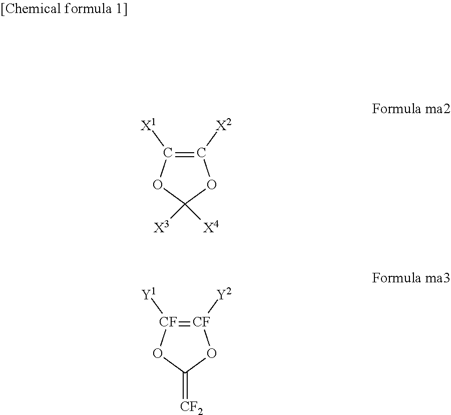

In the formulas, X¹, X², X³, X⁴, Y¹ and Y² each independently represent a fluorine atom, a perfluoroalkyl group which may have an etheric oxygen atom, or a perfluoroalkoxy group which may have an etheric oxygen atom, and X³ and X⁴ may be bonded together to form a ring.

In the formula ma2 and the formula ma3, the perfluoroalkyl group for X¹, X², X³, X⁴, Y¹ or Y² preferably has 1 to 7 carbon atoms, more preferably 1 to 5 carbon atoms, and particularly preferably 1 to 4 carbon atoms. The perfluoroalkyl group is preferably linear or branched, and a linear group is particularly desirable. The perfluoroalkyl group is preferably a trifluoromethyl group, pentafluoroethyl group, or heptafluoropropyl group or the like, and a trifluoromethyl group is particularly desirable.

Examples of the perfluoroalkoxy group for X¹, X², X³, X⁴, Y¹ or Y² include groups in which an oxygen atom (—O—) is bonded to one of the above perfluoroalkyl groups, and a trifluoromethoxy group is particularly desirable.

When the number of carbon atoms in the perfluoroalkyl group or perfluoroalkoxy group is two or greater, an etheric oxygen atom (—O—) may be interposed between carbon atoms of the perfluoroalkyl group or perfluoroalkoxy group.

In the formula ma2, X¹ is preferably a fluorine atom.

X² is preferably a fluorine atom, a trifluoromethyl group or a perfluoroalkoxy group of 1 to 4 carbon atoms, and a fluorine atom or a trifluoromethoxy group is particularly desirable.

X³ and X⁴ preferably independently each represent a fluorine atom or a perfluoroalkyl group of 1 to 4 carbon atoms, and a fluorine atom or a trifluoromethyl group is particularly desirable.

X³ and X⁴ may be bonded together to form a ring. The number of atoms constituting the ring skeleton of this ring is preferably from 4 to 7 atoms, and more preferably 5 or 6 atoms.

In the formula ma3, Y¹ and Y² preferably independently each represent a fluorine atom, a perfluoroalkyl group of 1 to 4 carbon atoms, or a perfluoroalkoxy group of 1 to 4 carbon atoms, and a fluorine atom or a trifluoromethyl group is particularly desirable.

Specific examples of preferred compounds for the compound ma2 include compounds ma21 to ma25 shown below.

Specific examples of preferred compounds for the compound ma3 include compounds ma31 and ma32 shown below.

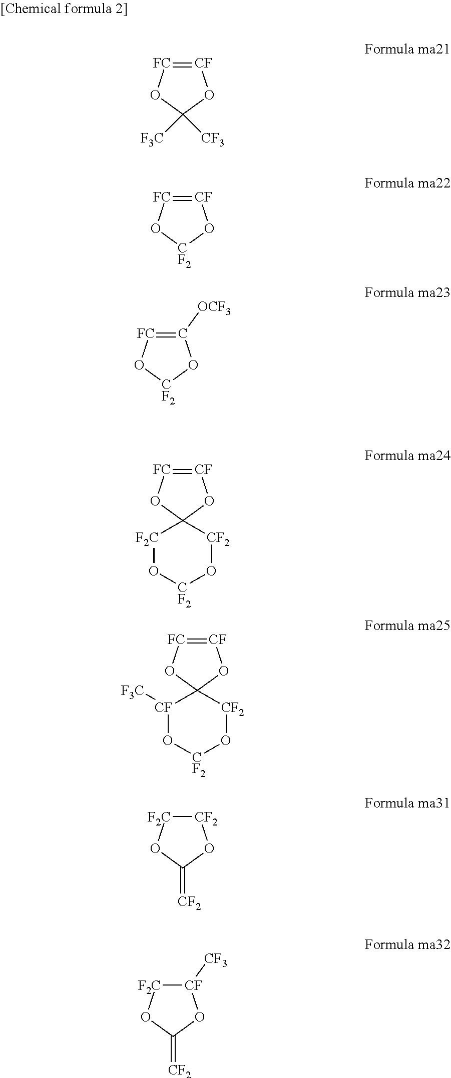

The unit a1 is preferably at least one type of unit selected from the group consisting of units a11 to a16 described below.

The units a11 to a14 are units formed by cyclopolymerization of the compound ma1. At least one of the units among a11 to a14 is produced by cyclopolymerization of the compound ma1. At that time, among the units a11 to a14, those units having a structure in which the number of atoms that constitute the ring skeleton of the fluorine-containing aliphatic ring is either 5 or 6 are produced most readily. A polymer containing two or more types of these units is sometimes produced.

In other words, a compound ma1 having a structure in which, within the units a11 to a14 described below, the number of atoms constituting the ring skeleton, including the atoms within Q, is either 5 or 6 is preferred.

A unit a15 shown below is a unit formed from the compound ma2, and a unit a16 described below is a unit formed from the compound ma3.

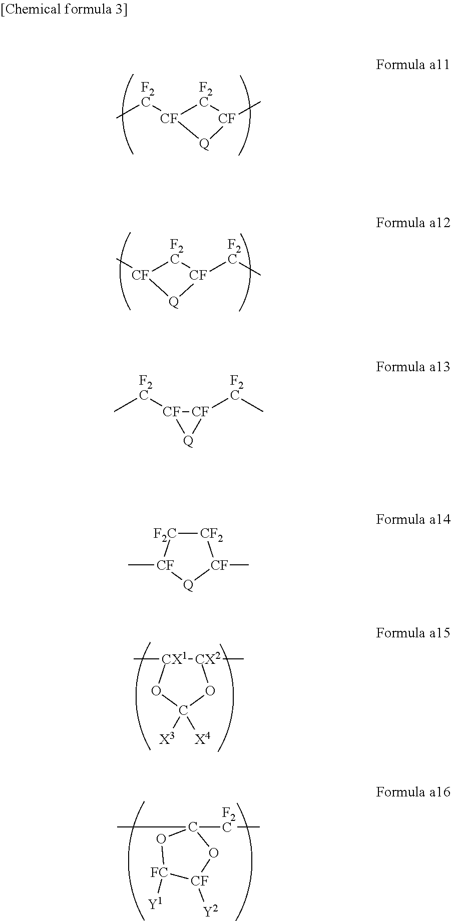

In terms of exhibiting superior chemical stability, the unit a1 is preferably a unit formed by cyclopolymerization of a diene-based fluorine-containing polymer.

The unit a1 in the polymer A may be a single type of unit, or a combination of two or more types of units.

The amount of the unit a1 within the polymer A, relative to the total of all the units that constitute the polymer A, is preferably at least 30 mol %, more preferably at least 50 mol %, even more preferably at least 70 mol %, and may be 100 mol %. Provided the amount of the unit a1 is at least as large as the above lower limit, the polymer exhibits particularly superior transparency and solvent solubility.

The polymer A may, if necessary, also have a unit other than the unit a1 (hereinafter also referred to as a “unit a2”).

There are no particular limitations on the unit a2, which may be any unit based on a monomer that can undergo copolymerization with the monomer that forms the unit a1. Examples of the unit a2 include units based on a monomer having a reactive functional group and a polymerizable double bond (hereinafter also referred to as a “unit a21”), units based on a fluorine-containing olefin such as tetrafluoroethylene, and units based on a fluorine-containing vinyl ether. The unit a2 in the polymer A may be a single type of unit, or a combination of two or more types of units.

Examples of the polymerizable double bond include CF₂═CF—, CF₂═CH—, CH₂═CF—, CFH═CF—, CFH═CH—, CF₂═C—, and CF═CF—.

The reactive functional group means a group having reactivity which, when drying or the like is conducted, undergoes reaction and enables the formation of chemical bonds (hydrogen bonds or covalent bonds or the like) between molecules of the polymer having the reactive functional group, or between the polymer and another substance other than the polymer (such as a metal or alloy). Examples of the reactive functional group include a carboxyl group, acid halide group, alkoxycarbonyl group, carbonyloxy group, carbonate group, sulfo group, phosphono group, hydroxyl group, thiol group, silanol group, and coupling group. The unit a21 may also have an ester group or an amide group.

Excluding the unit a21, the unit a2 is preferably a perfluoro unit. In the case of the unit a21, excluding the portion having the reactive functional group, the unit preferably has no hydrogen atoms bonded to carbon atoms.

An example of the unit a21 is a unit a21-1.

The unit a21-1 can be formed by polymerizing CF₂═CF—O—R^{f}—X.

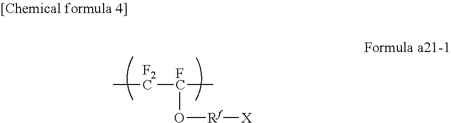

In the formula, R^{f} represents a perfluoroalkylene group which may have an etheric oxygen atom, and X represents COOH, COOR, SO₂F, SO₃R or SO₃H, wherein R represents an alkyl group of 1 to 5 carbon atoms.

In formula a21-1, the perfluoroalkylene group for R^{f} is preferably either linear or branched. The number of carbon atoms in the perfluoroalkylene group is preferably within a range from 2 to 10, more preferably from 2 to 7, and particularly preferably from 2 to 5.

The perfluoroalkylene group may have an etheric oxygen atom. In such cases, the number of etheric oxygen atoms within the perfluoroalkylene group may be either one, or two or more.

Specific examples of R^{f} include the groups shown below.

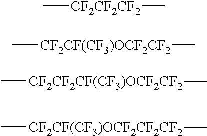

The alkyl group for R in COOR or SO₃R is preferably linear or branched. The alkyl group is preferably an alkyl group of 1 to 6 carbon atoms, and a methyl group is particularly desirable.

X is preferably COOCH₃, SO₂F, COOH or SO₃H.

In the unit a21-1, the portion in the formula represented by —O—R^{f}—X is preferably one of the following.

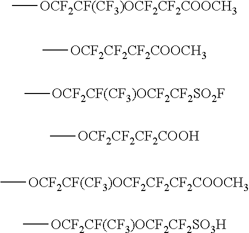

The polymer A may have a functional group at a terminal of the main chain. Examples of the functional group include an alkoxycarbonyl group (hereinafter also referred to as an “ester group”) having an alkoxy group of 1 to 3 carbon atoms, and amide group having a coupling group, a carboxyl group, and a carbonic acid fluoride group.

The alkoxy group in the ester group may be either linear or branched. The number of carbon atoms in the alkoxy group is preferably one or two, and most preferably one. Accordingly, a methoxycarbonyl group or ethoxycarbonyl group is preferred as the ester group, and a methoxycarbonyl group is particularly desirable.

Including an amide group having a coupling group results in excellent adhesion.

Examples of the coupling group include a silane coupling group or a phenol group or the like. In terms of achieving superior adhesion, a silane coupling group is preferred.

In terms of offering particularly superior adhesion to the substrate, the coupling group is preferably a group 2.

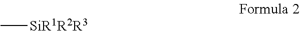

Each of R¹, R² and R³ independently represents an alkoxy group of 1 to 3 carbon atoms or an alkyl group of 1 to 3 carbon atoms, provided that at least one of R¹, R² and R³ represents an aforementioned alkoxy group.

For R¹, R² and R³, the alkoxy group may be either linear or branched. The alkyl group may also be either linear or branched.

By ensuring that at least one of R¹, R² and R³ represents an alkoxy group, the adhesion with the substrate is more superior. The number of alkoxy groups among R¹, R² and R³ is preferably two or three.

Specific examples of SiR¹R²R³ include a methyldiethoxysilyl group, methyldimethoxysilyl group, trimethoxysilyl group, and triethoxysilyl group.

Examples of the amide group having a group 2 include the group 3 shown below.

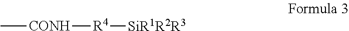

R¹, R² and R³ are as defined above for R¹, R² and R³ in formula 2, and R⁴ represents an alkylene group, a group in which an imino group is interposed between carbon atoms of an alkylene group, or an arylene group.

The alkylene group for R⁴ may be either linear or branched. The number of carbon atoms in the alkylene group is preferably within a range from 1 to 10, and more preferably from 1 to 4. In the case of a group in which an imino group is interposed between carbon atoms of an alkylene group, the number of carbon atoms in the alkylene group is preferably within a range from 2 to 10, and the number of imino groups is preferably within a range from 1 to 3. The number of carbon atoms in the arylene group is preferably within a range from 6 to 10.

Specific examples of R⁴ include —(CH₂)₃—, —(CH₂)₂NH(CH₂)₃—, and a phenylene group.

The weight average molecular weight (Mw) of the fluororesin is preferably within a range from 10,000 to 500,000, and more preferably from 30,000 to 200,000. Provided Mw is at least a large as the above lower limit, the toughness of the fluororesin is superior, whereas provided Mw is not more than the above upper limit, the solubility in solvents and the moldability are superior.

A commercially available product may be used as the fluororesin, or a fluororesin produced using conventional methods may be used. Examples of preferred commercially available fluororesins include CYTOP (a registered trademark) manufactured by AGC Inc. (a cyclopolymer of perfluoro (3-butenyl vinyl ether), Teflon (a registered trademark) AF manufactured by Chemours Co., Ltd. (a tetrafluoroethylene/perfluoro (2,2-dimethyl-1,3-dioxole) copolymer), Hyflon (a registered trademark) AD manufactured by Solvay S. A. (a tetrafluoroethylene/perfluoro (4-methoxy-1,3-dioxole) copolymer), and FluoroPel PFC1601 and PFC1101V manufactured by Cytonix Corporation.

One of these fluororesins may be used alone, or a combination of two or more such resins may be used.

### <Non-Fluororesin>

A non-fluororesin is a resin having no fluororesin.

Conventional non-fluororesins may be used as the non-fluororesin, provided the resin is capable of forming a porous layer.

Examples of preferred non-fluororesins include crystalline non-fluororesins. A crystalline non-fluororesin is a non-fluororesin which, when solidified by lowering the temperature, has a crystalline portion in which the molecules are aligned in a regular manner. By including a crystalline non-fluororesin in the fibers that constitute the nonwoven fabric, the chemical resistance and mechanical strength can be further improved.

Examples of the non-fluororesin include polycaprolactone, polyethylene glycol, polyvinyl alcohol, polyacrylonitrile, polyaniline, acrylic resins, polyurethane, polystyrene, polycarbonate, polyvinylpyrrolidone, chitosan, gelatin and cellulose acetate.

Examples of crystalline non-fluororesins include polycaprolactone, polyethylene glycol, polyacrylonitrile, polyaniline, chitosan, gelatin and cellulose acetate.

Among these, in terms of achieving particularly superior moldability and mechanical strength, polycaprolactone and polyacrylonitrile are preferred.

One of these non-fluororesins may be used alone, or a combination of two or more such resins may be used.

### (Substrate)

The substrate may be any type of conventional material typically used as the substrate of a microfluidic device, and examples include glass substrates, polycarbonate, polyimide, and silicon wafers. The thickness of the substrate is, for example, within a range from 0.01 to 10 mm.

One or more other layers may be formed on the surface of the substrate.

Examples of these other layers include electrodes and dielectric layers other than the porous layer. Electrodes and a dielectric layer other than the porous layer may be formed in that order on the substrate.

Examples of the electrode material include metals, metal mixtures, metal-semiconductor mixtures, and conductive resins. The thickness of the electrode is, for example, typically within a range from 0.001 to 10 mm.

Examples of dielectric layers other than the porous layer include layers of dielectrics (such as parylene C) formed by CVD methods, and layers of dielectrics (for example, fluororesins such as ethylene-tetrafluoroethylene copolymers) formed by wet coating methods such as spin-coating. The thickness of such dielectric layers other than the porous layer is, for example, within a range from 0.1 to 1,000 μm.

### [Uses]

There are no particular limitations on the potential uses for the microfluidic device, and examples of possible uses include biochips, clinical real-time diagnosis of diseases, biosensors, cell culturing (for example, supply of insulin or penicillin to a medium), drug delivery applications, FDA applications relating to food safety, microfluidic fuel cells and energy applications, and microfluidic reactors (for example, synthesis of lipid nanoparticles (LNP)).

Examples of biochip applications include enzyme analyses (for example, glucose or lactic acid assays), DNA analyses (for example, PCR or high-throughput sequencing), DNA cloning, antibody purification, production and analyses, immunological testing, and proteome analyses. The term “proteome” is a coined term meaning a combination of a protein and a genome. Whereas a genome indicates the entire genetic information of a single organism, a proteome indicates total protein expressed (capable of being expressed) intracellularly. In those cases where the microfluidic device is used in a biochip application, it is desirable that the liquid repellency of the biochip is maintained even after contact for a certain time with the various biotest liquids being used.

### [Production Method for Microfluidic Device]

The production method for a microfluidic device according to one aspect of the present invention has a step of spinning core-shell fibers using an electrospinning method and depositing the fibers on a substrate to form a porous layer (an electrospinning step). In the electrospinning step, a layer of a nonwoven fabric composed of core-shell fibers is formed as the porous layer.

### (Electrospinning Step)

The electrospinning step may be conducted using conventional methods.

One example of the electrospinning step is described below with reference to FIG. 2.

FIG. 2 is a schematic structural diagram illustrating one example of an electrospinning device used in the production of a layer of a nonwoven fabric composed of fibers having a core-shell structure.

The electrospinning device of this example includes a needle-like nozzle 31, a first syringe pump 32 that supplies a core solution to the nozzle 31, and a second syringe pump 33 that supplies a shell solution to the nozzle 31. The electrospinning device may also include a power source 34 that applies a voltage to the core solution and the shell solution at the tip of the nozzle 31, and a collector 35.

The nozzle 31 is a core-shell coaxial spinneret, and includes an outer cylinder and an inner cylinder disposed coaxially inside the outer cylinder. The position of the tip of the inner cylinder and the position of the tip of the outer cylinder along the axial direction are approximately equal. The inside of the peripheral edge of the tip of the inner cylinder functions as the core solution discharge port, and the space between the peripheral edge of the tip of the inside cylinder and the peripheral edge of the tip of the outer cylinder functions as the shell solution discharge port. The shell solution discharge port surrounds the core solution discharge port in a ring shape.

The length in the axial direction of the inner cylinder is longer than that of the outer cylinder, so that the base end of the inner cylinder protrudes from the outer cylinder. The first syringe pump 32 is connected to the base end of the inner cylinder, and by operating the first syringe pump 32, the core solution is supplied to the inside of the inner cylinder and is discharged from the opening at the tip (namely, the core solution discharge port). The second syringe pump 33 is connected to the base end of the outer cylinder, and by operating the second syringe pump 33, the shell solution is supplied into the outer cylinder and forced out of the opening at the tip (namely, the shell solution discharge port).

In the production of a nonwoven fabric using the electrospinning device of this example, the substrate is placed on top of the collector 35, the core solution and the shell solution are supplied to the nozzle 31, and a voltage is applied.

When the dispersion force generated by the charge on the solution surface exceeds the surface tension, the core solution and shell solution forced from the nozzle 31 form a cone shape known as a Taylor cone at the tip of the nozzle 31, which extends down toward the collector 35. This flow of extended solution narrows and becomes unstable as the solvent evaporates, forming a whipping state in which an expanded state is generated from the single narrow stream. Depending on conditions, the flow may divide further. This flow is deposited on top of the substrate, forming a layer of a nonwoven fabric.

The core solution is a solution composed of the resin for forming the core dissolved in a solvent. The shell solution is a solution composed of the resin for forming the shell dissolved in a solvent.

Preferred forms of the core and shell are as described above.

The resin concentration in the core solution, for example, relative to the total mass of the core solution, is within a range from 1 to 20% by mass.

The resin concentration in the shell solution, for example, relative to the total mass of the shell solution, is within a range from 1 to 20% by mass.

The solvent of the core solution may be any solvent capable of dissolving the resin that forms the core, and may be selected appropriately from among a11 manner of conventional solvents. This also applies to the solvent of the shell solution.

Examples of the solvents include protic solvents and aprotic solvents. A “protic solvent” is a solvent that has a proton donor ability. An “aprotic solvent” is a solvent that does not have a proton donor ability.

Examples of protic solvents include the protic fluorine-containing solvents listed below.

Namely, specific examples of protic fluorine-containing solvents include fluorine-containing alcohols such as trifluoroethanol, 2,2,3,3,3-pentafluoro-1-propanol, 2-(perfluorobutyl) ethanol, 2-(perfluorohexyl) ethanol, 2-(perfluorooctyl) ethanol, 2-(perfluorodecyl) ethanol, 2-(perfluoro-3-methylbutyl) ethanol, 2,2,3,3,-tetrafluoro-1-propanol, 2,2,3,3,4,4,5,5-octafluoro-1-pentanol, 2,2,3,3,4,4,5,5,6,6,7,7-dodecafluoro-1-heptanol, 2,2,3,3,4,4,5,5,6,6,7,7,8,8-hexadecafluoro-1-nonanol, 1,1,1,3,3,3-hexafluoro-2-propanol, and 1,3,3,4,4,4-hexafluoro-2-butanol; fluorine-containing carboxylic acids such as trifluoroacetic acid, perfluoropropanoic acid, perfluorobutanoic acid, perfluoropentanoic acid, perfluorohexanoic acid, perfluoroheptanoic acid, perfluorooctanoic acid, perfluorononanoic acid, perfluorodecanoic acid, 1,1,2,2-tetrafluoropropanoic acid, 1,1,2,2,3,3,4,4-octafluoropentanoic acid, 1,1,2,2,3,3,4,4,5,5-dodecafluoroheptanoic acid, and 1,1,2,2,3,3,4,4,5,5,6,6-hexadecafluorononanoic acid; and fluorine-containing sulfonic acids such as trifluoromethanesulfonic acid and heptadecafluorooctanesulfonic acid.

Examples of aprotic solvents include the aprotic fluorine-containing solvents listed below.

Namely, specific examples of aprotic fluorine-containing solvents include polyfluoro aromatic compounds such as 1,4-bis(trifluoromethyl)benzene, polyfluorotrialkylamine compounds such as perfluorotributylamine, polyfluorocycloalkane compounds such as perfluorodecalin, polyfluoro cyclic ether compounds such as perfluoro (2-butyltetrahydrofuran), as well as perfluoropolyethers, polyfluoroalkane compounds, and hydrofluoroethers (HFE).

One of these solvents may be used alone, or a combination of two or more solvents may be used. Further, a wide range of compounds besides those listed above may also be used.

The supply volume of the core solution to the nozzle 31 per unit of time (hereinafter also referred to as the “core solution flow rate”) is preferably within a range from 0.1 to 10 mL/hour, and more preferably from 0.3 to 3 mL/hour.

The supply volume of the shell solution to the nozzle 31 per unit of time (hereinafter also referred to as the “shell solution flow rate”) is preferably within a range from 0.5 to 15 mL/hour, and more preferably from 1 to 6 mL/hour.

The ratio represented by (shell solution flow rate)/(core solution flow rate) (hereinafter also referred to as the “shell/core flow rate ratio”) is preferably within a range from 0.5/10 to 15/0.1, and more preferably from ⅓ to 6/0.3.

The separation distance between the tip of the nozzle 31 and the collector 35 is, for example, within a range from 50 to 250 mm.

The applied voltage is, for example, within a range from 5 to 35 kV.

By appropriate adjustment of factors such as the resin concentration and flow rate of the core solution, the resin concentration and flow rate of the shell solution, the applied voltage, and the distance between the tip of the nozzle 31 and the collector 35, the diameter of the fibers that constitute the nonwoven fabric, and the surface roughness and density and the like of the nonwoven fabric layer can be altered.

Following the electrospinning step, the laminate composed of the porous layer laminated on top of the substrate obtained in the electrospinning step can be used to assemble a microfluidic device. In such cases, following the electrospinning step but prior to assembly of the microfluidic device, the laminate may be heated or subjected to thermal compression to improve the uniformity of the porous layer and the adhesion to the substrate.

Following the electrospinning step, the porous layer on the substrate may also be transferred to a separate substrate (hereinafter also referred to as a “transfer step”), and the laminate obtained in this transfer step then used to assemble a microfluidic device. In those cases where a transfer step is conducted, the collector 35 may be used as the substrate in the electrospinning step. In those cases where a transfer step is conducted, the laminate composed of the porous layer laminated on top of the substrate may be heated or subjected to thermal compression prior to transfer to improve the uniformity of the porous layer. In those cases where a transfer step is conducted, following the transfer step but prior to assembly of the microfluidic device, the laminate may be heated or subjected to thermal compression to improve the uniformity of the porous layer and the adhesion to the substrate.

The method used for assembling the microfluidic device using the laminate may employ the same method as that used for conventional microfluidic devices, with the exception of using the laminate described above for at least a portion of the structural members constituting the microfluidic device.

### EXAMPLES

The present invention is described below in further detail using a series of examples and comparative examples, but the present invention is not limited to the following examples, provided it does not exceed the scope of the invention. Examples 1 to 4 are examples of the invention, and Examples 5 and 6 are comparative examples.

### (Measurement Methods)

### <Fiber Diameter>

The diameters of the fibers constituting the nonwoven fabric were measured using a laser microscope VK-8710 (manufactured by Keyence Corporation).

### <Nonwoven Fabric Thickness>

The thickness of the nonwoven fabric was measured using a laser microscope VK-8710 (manufactured by Keyence Corporation).

### <Initial Water Contact Angle>

The water contact angle of a distilled water droplet of approximately 2 μL placed on the nonwoven fabric surface of the laminate (or on the fluororesin layer surface in Examples 5 and 6) was measured using a contact angle meter DropMaster DMo-701SA (manufactured by Kyowa Interface Science Co., Ltd.). The water contact angle was measured at 5 different locations on the surface of the surface layer, and the mean value of the measurements was calculated and recorded as the water contact angle. The 2θ method was used in the contact angle calculations.

### <Bio Test Liquid Tolerance Test>

One hundred μL of a protein standard substance (“Analytical Standard 200 mg/mL BSA” manufactured by Sigma-Aldrich Co., Ltd.) was placed on the surface of the laminate and left to stand for one hour. After one hour, the protein standard substance was wiped away with a dry towel, the surface was then washed with distilled water and dried under a flow of nitrogen, and the water contact angle was measured using the method described above in the section entitled <Initial Water Contact Angle> and recorded as the post-test water contact angle. A post-test water contact angle closer to the initial water contact angle (a smaller change in the water contact angle) was used to confirm superior bio test liquid tolerance. A difference between the initial water contact angle and the post-test water contact angle of 56° or less was evaluated as indicating superior bio test liquid tolerance.

### (Materials Used)

Fluororesin solution-1: a solution of a perfluoro (3-butenyl vinyl ether) cyclopolymer (CTL-107MK manufactured by AGC Inc., solid fraction: 7% by mass, solvent: CT-SOLV100E manufactured by AGC Inc.).

Fluororesin solution-2: a solution of a perfluoro (3-butenyl vinyl ether) cyclopolymer (CTL-809M manufactured by AGC Inc., solid fraction: 9% by mass, solvent: CT-SOLV180 manufactured by AGC Inc.).

Fluororesin solution-3: a solution of a tetrafluoroethylene/perfluoro (2,2-dimethyl-1,3-dioxole) copolymer (Teflon (a registered trademark) AF 1604 manufactured by Chemours Co., Ltd., solid fraction: 4% by mass, solvent: FC40 manufactured by 3M Co., Ltd.).

PCL solution: a solution of a polycaprolactone with a number average molecular weight (Mn) of 70,000 to 90,000 (solid fraction: 10% by mass, solvent: trifluoroethanol).

### Examples 1 to 4

An electrospinning device was prepared with the structure illustrated in FIG. 2.

A glass plate was placed on the collector 35 of the electrospinning device as a substrate.

By setting the types of core solution and shell solution, the flow rates for the core solution and the shell solution, the separation distance between the tip of the nozzle 31 and the collector 35, and the electrospinning voltage value as illustrated in Table 1, and then conducting electrospinning under conditions including a temperature of 29° C. and a humidity of 35% RH, core-shell fibers were deposited on the substrate, yielding a laminate having a nonwoven fabric layer laminated on top of the substrate as a porous layer. The diameter of the fibers constituting the nonwoven fabric, the thickness of the nonwoven fabric, and the initial water contact angle for the surface of the nonwoven fabric are also shown in Table 1.

The laminate of Example 4 was subjected to the bio test liquid tolerance test. The post-test water contact angle was 128°.

TABLE 1

| | | | | | Distance | | | | |
|---|---|---|---|---|---|---|---|---|---|
| | | | | | between | | | | |
| | | | Flow rate | | nozzle tip | | Nonwoven fabric | | |
| | | | (mL/h) | | and | | Fiber | | Initial water |
| | Core | Shell | Core | Shell | collector | Voltage | diameter | Thickness | contact angle |
| Example | solution | solution | solution | solution | (mm) | (kV) | (nm) | (μm) | (°) |
| 1 | Fluororesin | PCL | 0.5 | 3 | 150 | 16 | 440 | 70 | 132.2 |
| | solution-1 | solution | | | | | | | |
| 2 | PCL | Fluororesin | 1 | 3 | 150 | 14 | 470 | 70 | 133.8 |
| | solution | solution-1 | | | | | | | |
| 3 | Fluororesin | PCL | 1 | 3 | 150 | 18 | 500 | 70 | 130.7 |
| | solution-2 | solution | | | | | | | |
| 4 | Fluororesin | PCL | 0.5 | 3 | 150 | 18 | 520 | 70 | 152.1 |
| | solution-2 | solution | | | | | | | |

### Example 5

The fluororesin solution-3 was applied to a substrate (glass) using a spin coater under conditions including a spin rate of 4,000 rpm and an application period of 20 seconds, and the resulting laminate was then baked at 330° C. for 60 minutes, yielding a laminate composed of a fluororesin layer that was not a porous layer laminated on top of the substrate. The initial water contact angle of the surface of the fluororesin layer was 121°. Further, a bio test liquid tolerance test was then conducted. The post-test water contact angle was 64°.

### Example 6

The fluororesin solution-2 was applied to a substrate (glass) using a spin coater under conditions including a spin rate of 4,000 rpm and an application period of 10 seconds, and the resulting laminate was subjecting to preliminary drying at 60° C. for 10 minutes and then baking at 190° C. for 60 minutes, yielding a laminate composed of a fluororesin layer that was not a porous layer laminated on top of the substrate. The initial water contact angle of the surface of the fluororesin layer was 115°. Further, a bio test liquid tolerance test was then conducted. The post-test water contact angle was 55°.

As is evident from the above results, the surfaces on the side of the nonwoven fabrics of the laminates of Examples 1 to 4 exhibited higher initial water contact angles and superior liquid repellency compared with the surfaces of the fluororesin layers of the laminates of Examples 5 and 6. Further, the nonwoven fabric surface of the laminate of Example 4 exhibited superior bio test liquid tolerance compared with the surfaces on the side of the fluororesin layers of the laminates of Examples 5 and 6.

### INDUSTRIAL APPLICABILITY

The microfluidic device of the present invention exhibits excellent liquid repellency, and can be used, for example, in biochips (enzyme analyses (for example, glucose or lactic acid assays), DNA analyses (for example, PCR or high-throughput sequencing), DNA cloning, antibody purification, production and analyses, immunological testing, and proteome analyses), clinical real-time diagnosis of diseases, biosensors, cell culturing (for example, supply of insulin or penicillin to a medium), drug delivery applications, FDA applications relating to food safety, microfluidic fuel cells and energy applications, and microfluidic reactors (for example, synthesis of lipid nanoparticles (LNP)).

### REFERENCE SIGNS LIST

1: First laminate; 2: Second laminate; 11: First substrate; 12: Electrode; 13: First dielectric layer; 21: Second substrate; 22: Common electrode; 23: Second dielectric layer; L: Liquid droplet; 31: Nozzle; 32: First syringe pump; 33: Second syringe pump; 34: Power source; 35: Collector.

What is claimed is:

1. A microfluidic device comprising:
a laminate having a porous layer laminated on a substrate.

2. The microfluidic device according to claim 1, wherein the porous layer is a layer of a nonwoven fabric.

3. The microfluidic device according to claim 2, wherein the nonwoven fabric is a nonwoven fabric formed using an electrospinning method.

4. The microfluidic device according to claim 2, wherein a fiber that constitutes the nonwoven fabric contains a fluororesin.

5. The microfluidic device according to claim 4, wherein the fiber that constitutes the nonwoven fabric further contains a non-fluororesin.

6. The microfluidic device according to claim 2, wherein the fiber that constitutes the nonwoven fabric has a core-shell structure.

7. The microfluidic device according to claim 6, wherein one of core and shell of the core-shell structure contains a fluororesin, and the other contains a non-fluororesin.

8. The microfluidic device according to claim 4, wherein the fluororesin is an amorphous fluororesin.

9. The microfluidic device according to claim 4, wherein the fluororesin contains a polymer having a unit having a fluorine-containing aliphatic cyclic structure that constitutes a main chain.

10. The microfluidic device according to claim 9, wherein the unit having a fluorine-containing aliphatic cyclic structure that constitutes the main chain is at least one type of unit selected from the group consisting of units formed by cyclopolymerization of a diene-based fluorine-containing monomer, and units based on a cyclic fluorine-containing monomer.

11. The microfluidic device according to claim 5, wherein the non-fluororesin is a crystalline non-fluororesin.

12. The microfluidic device according to claim 5, wherein the non-fluororesin is at least one type of resin selected from the group consisting of polycaprolactone, polyethylene glycol, polyvinyl alcohol, polyacrylonitrile, polyaniline, acrylic resins, polyurethane, polystyrene, polycarbonate, polyvinylpyrrolidone, chitosan, gelatin and cellulose acetate.

13. The microfluidic device according to claim 1, wherein the device is a biochip.

14. A production method for a microfluidic device, the method comprising:
spinning a fiber having a core-shell structure using an electrospinning method and depositing the fiber on a substrate to form a porous layer.

15. The production method according to claim 14, wherein one of core and shell of the core-shell structure contains a fluororesin, and the other contains a non-fluororesin.

16. The production method according to claim 15, wherein the fluororesin is an amorphous fluororesin.

17. The production method according to claim 15, wherein the fluororesin contains a polymer having a unit having a fluorine-containing aliphatic cyclic structure that constitutes a main chain.

18. The production method according to claim 17, wherein the unit having a fluorine-containing aliphatic cyclic structure that constitutes the main chain is at least one type of unit selected from the group consisting of units formed by cyclopolymerization of a diene-based fluorine-containing monomer, and units based on a cyclic fluorine-containing monomer.

19. The production method according to claim 15, wherein the non-fluororesin is a crystalline non-fluororesin.

20. The production method according to claim 15, wherein the non-fluororesin is at least one type of resin selected from the group consisting of polycaprolactone, polyethylene glycol, polyvinyl alcohol, polyacrylonitrile, polyaniline, acrylic resins, polyurethane, polystyrene, polycarbonate, polyvinylpyrrolidone, chitosan, gelatin and cellulose acetate.
